(19) United States
(12) Patent Application Publication
(10) Pub. No.: US 2009/0082802 A1
(43) Pub. Date: Mar. 26, 2009

(54) Mechanism and Method for Closing an Arteriotomy

(75) Inventors: Thierry Benjamin, Lowell, MA (US); David Yann, Lynn, MA (US); Matthew Spurchise, Peabody, MA (US); Juan-Pablo Mas, Somerville, MA (US); Richard Rego, Mansfield, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/861,726

(22) Filed: Sep. 26, 2007

(51) Int. Cl. A61B 17/08

(52) U.S. Cl. 606/213

(57) ABSTRACT

Devices and methods for closing an arteriotomy by gripping surfaces of tissue disposed about the arteriotomy, without piercing the tissue, and drawing the gripped tissue together to approximate the edges of the arteriotomy, and, while holding the tissue, applying a more permanent closure to the tissue.

### FIELD OF THE INVENTION

The invention relates to devices and techniques for closing a percutaneous puncture in a blood vessel, after an intravascular procedure and, particularly, to a mechanism and technique for temporarily holding the arteriotomy closed while a more permanent closure can be made.

### BACKGROUND

Various cardiovascular procedures, such as angioplasty, stent placement and atherectomy, among others, are performed by inserting into and manipulating within the vasculature, wires and catheters adapted to perform those procedures. Access to the vasculature typically is through the femoral artery and is percutaneous, involving insertion of a needle and introducer sheath in the region of the groin to form a track through subcutaneous tissue and to puncture and create an arteriotomy in the femoral artery. A guidewire then is advanced through the needle and into the femoral artery. The needle then is removed. An introducer sheath is then advanced over the guidewire, along the track and into the femoral artery. The wire and sheath provide access into the femoral artery, through the arteriotomy, for catheters or other instrumentalities in order to perform the selected procedure.

After the procedure has been completed, the procedural devices are removed and the arteriotomy must be closed. A number of techniques are known to facilitate closure and healing of the arteriotomy. These include application of pressure at the puncture site for a relatively extended length of time, or the use of biological adhesives or plugs adapted to seal the arteriotomy, or the use of staples or clips. Some closure systems include an arrangement to engage the artery to temporarily draw the edges of the arteriotomy together while a final closure device, such as a stapler, sutures, adhesives or other means is used to effect the permanent closure of the arteriotomy. Such systems are described, for example, in U.S. Pat. No. 6,767,356 (Kanner) and U.S. Pat. No. 6,391,048 (Ginn et al.). Ginn discloses an arrangement, in which several needles pierce the vessel wall surrounding the arteriotomy and then are manipulated to twist or draw together the vessel wall about the arteriotomy. Adhesives, sutures or clips then may be employed to secure a permanent closure.

It would be desirable to provide an alternate closure system in which tissue about the arteriotomy could be drawn together temporarily without risking the trauma from piercing the tissue. The present invention is directed to such an alternate mechanism and technique for closing the arteriotomy. The mechanism and technique lend themselves to the use of low profile devices.

### SUMMARY OF THE INVENTION

The invention employs a percutaneously placeable sheath mechanism deployable about an arteriotomy by which an array of tissue grippers having suction ports can be engaged with the surface of tissue about the arteriotomy enabling the ports to grip the tissue without piercing it. The tissue grippers can be deployed to a radially expanded configuration or a contracted configuration. The device may be advanced over an indwelling guidewire with the grippers in a radially contracted configuration. When the device is advanced to the region of the arteriotomy, the grippers then may be deployed to a more radially outward configuration about the region of the arteriotomy and, when in position, actuated to grip the tissue, as by applying suction to the suction ports. With the grippers holding the tissue, they are drawn radially inward together to purse the tissue together and close the arteriotomy. While maintaining the arteriotomy closed, the guidewire may be removed and a more permanent closure device can be advanced through the sheath to secure closure of the arteriotomy. The grippers then may be operated to release the tissue, as by terminating the suction, and the devices withdrawn.

### DESCRIPTION OF THE DRAWINGS

In the accompanying drawings:

FIG. 1 is a diagrammatic perspective of the mechanism at the distal end of the arteriotomy closure system as seen from a proximal oblique angle;

FIG. 2 is a diagrammatic perspective of the mechanism at the distal end of the arteriotomy closure system as seen from a distal oblique angle;

FIG. 3 is an illustration of the device with tissue-gripping retention fingers in a deployed position to be disposed against tissue about the arteriotomy;

FIG. 4 is an illustration of the device with tissue-gripping retention fingers in a radially contracted position to purse the tissue about the arteriotomy and close the arteriotomy;

FIG. 5 is an illustration in diagrammatic cross-section of the mechanism with the fingers radially contracted to purse the tissue about the arteriotomy and close the arteriotomy, with the guidewire having been removed;

FIGS. 6 and 6A are illustrations, in diagrammatic cross-section of the construction of the mechanism;

FIG. 7 is a diagrammatic illustration of another embodiment of the mechanism;

FIG. 8 is a sectional illustration of a control module at the proximal end of the device for communicating suction to the distal end and for enabling advancement or withdrawal of the closure tube with respect to the central tube;

FIGS. 9A-9D illustrate, diagrammatically, the manner in which the invention may be used to approximate the edges of an arteriotomy by engaging and drawing together connective tissue associated with the vessel in which the arteriotomy is formed;

FIGS. 10A and 10B are diagrammatic illustrations of the distal ends of modified central tubes;

FIG. 11 is a sectional illustration of a device in accordance with the invention used in association with a stabilizing system in engagement with the vessel; and

FIG. 12 is an illustration of another embodiment of a stabilizing system as may be used in connection with the invention.

### DESCRIPTION OF THE ILLUSTRATIVE EMBODIMENTS

The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

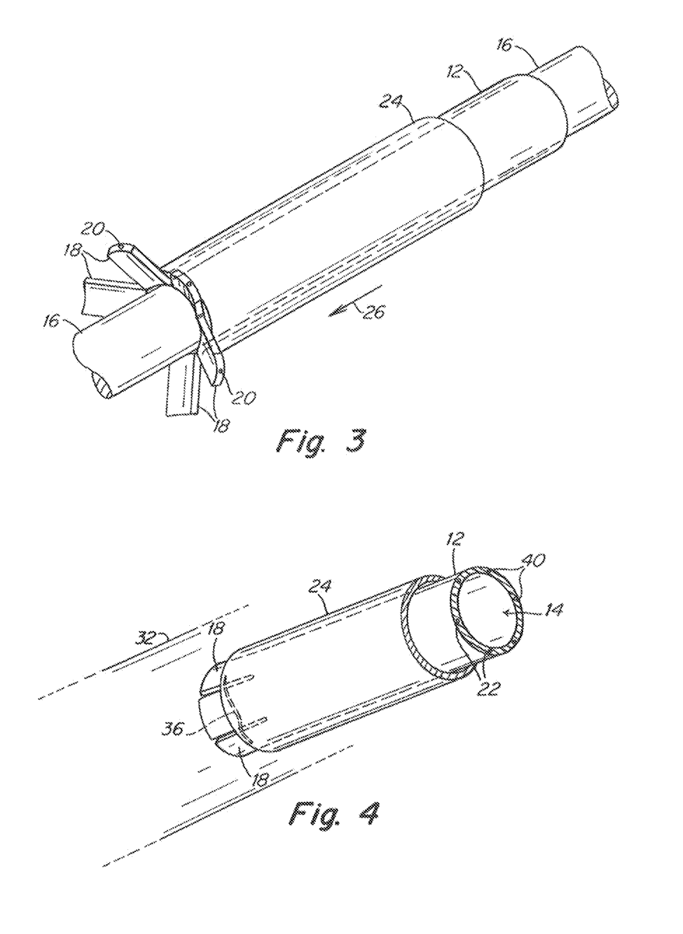

FIGS. 1-4 illustrate, diagrammatically, the distal portion of a mechanism 10 incorporating the invention, it being understood that the components of the mechanism 10 that are movable along an axial direction (i.e., along a guidewire) extend to the proximal end of the system where they may be controlled to perform their respective functions and movements at the distal, end. The mechanism 10, in this embodiment, includes a central tube 12 having a central lumen 14 adapted to receive a guidewire 16, as well as a closure device (not shown) when the guidewire 16 is removed. The distal end of the central tube 12 carries radially expandable and contractible elements, such as tissue retention fingers 18 that may be formed integrally with the central tube 12. The fingers 18 have gripping elements, such as suction ports 20, that do not puncture tissue, at their distal ends, the suction ports 20 being in communication with a source of suction (not shown) by suction lumens 22 extending proximally along the central tube 12.

The fingers 18 may be moved between a radially contracted, low profile, configuration (FIGS. 1, 2 and 4) or a radially expanded, deployed configuration (FIG. 3) by a closure tube 24 that is slidably disposed about the central tube. The fingers 18 may move in a generally radial pattern with or without symmetry about a central point, such as a central location of the arteriotomy. Alternatively, some or all of the fingers 18 may be adapted to expand and contract in a pattern having line symmetry. Thus, the terms “radial” or “radially” are not intended herein to be limited strictly to the radii of a circle when such terms are used to describe the movement of fingers 18.

The closure tube 24 extends to the proximal end of the device and can be advanced distally (arrow 26) to force the fingers 18 together in a contracted configuration, or may be withdrawn proximally (arrow 28) to allow the fingers to deploy to a radially expanded configuration. The fingers may be caused to deploy to a radially expanded configuration by forming them with a resilient bias toward the expanded configuration or by an arrangement of pull wires, or a combination of both. The fingers may be caused to flare outwardly to engage the surface of tissue about the arteriotomy and suction then can be applied to cause the fingers to grip that surface. The fingers then are drawn radially inwardly to pucker the tissue together and hold it in that configuration until a more permanent closure can be applied.

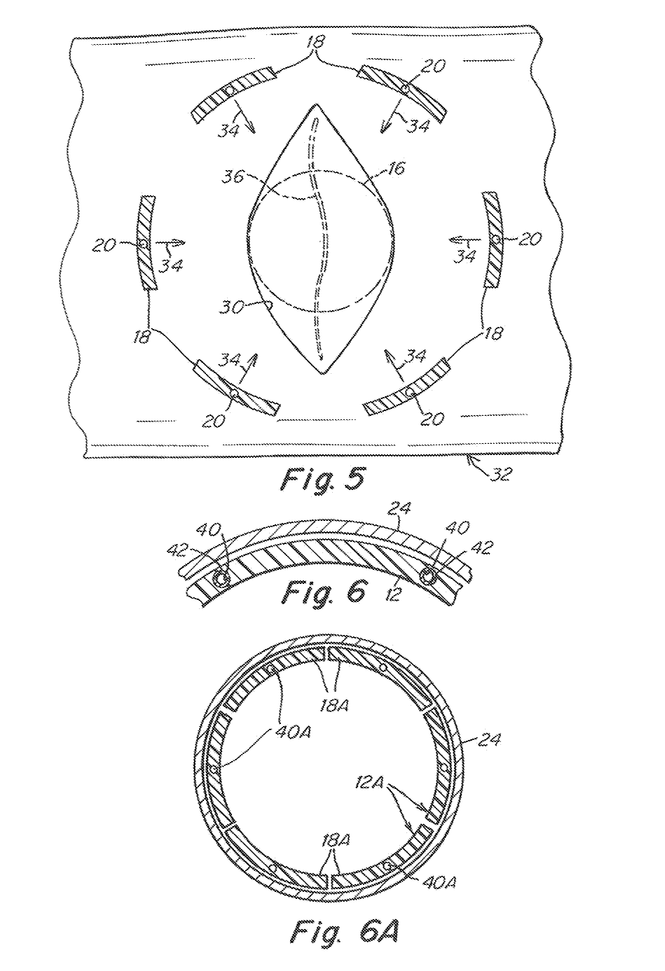

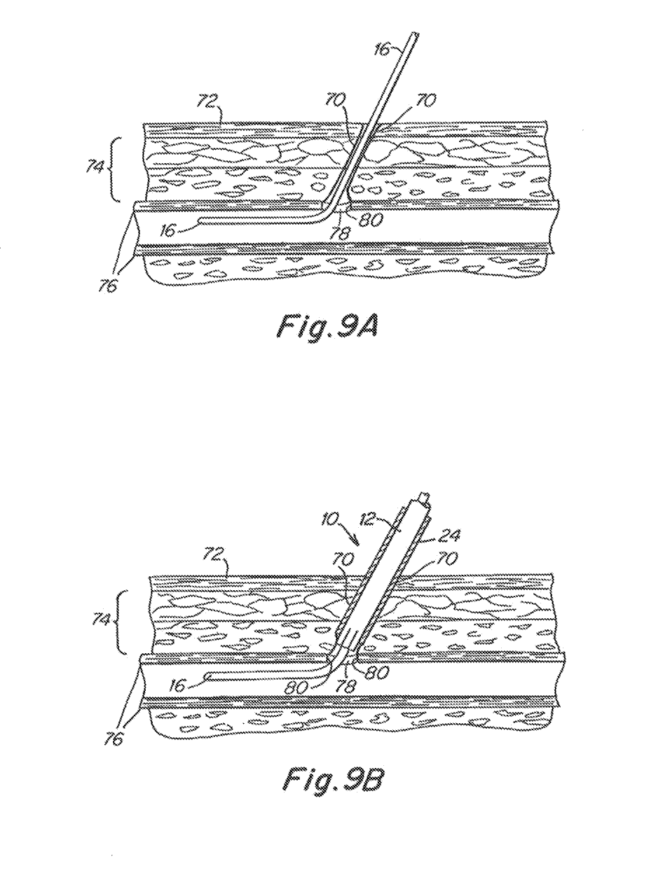

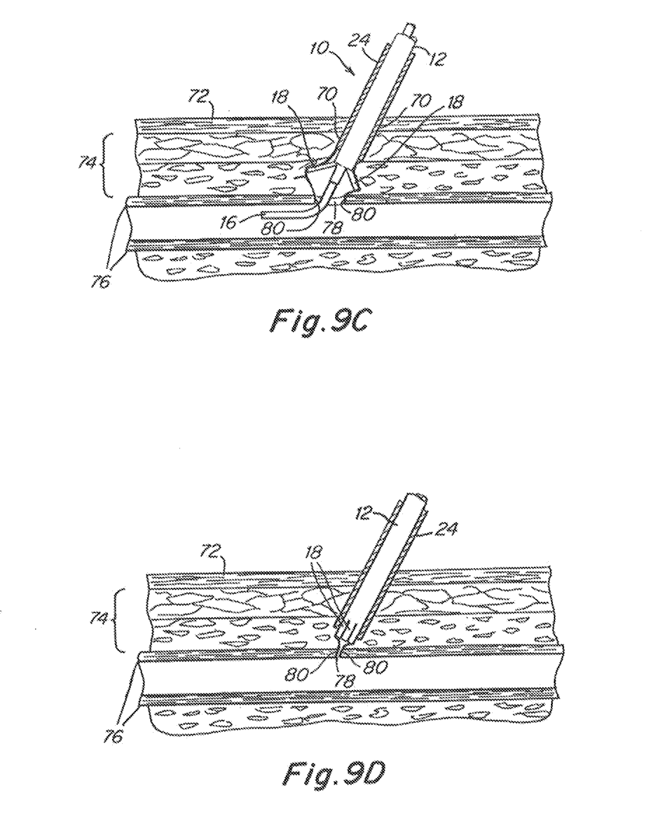

FIG. 5 illustrates, diagrammatically, the manner in which the mechanism may be used to purse an arteriotomy 30 together in readiness to be closed, as by a stapler, suturing device, adhesive or the like. The device is advanced along an indwelling guidewire 16 with the fingers 18 maintained in a contracted configuration as shown in FIGS. 1 and 9B to facilitate delivery of the device. When the distal end of the device is in proximity to the region of the arteriotomy, such as the tissue, of the femoral sheath that surrounds the artery 32, the fingers 18 are caused to deploy to their radially expanded configuration as suggested in FIG. 5 and are engaged with the tissue surface so that suction, applied through the suction lumens will enable the fingers to grip the tissue. With the tissue about the arteriotomy 30 so gripped, the mechanism is activated to draw the fingers together, radially inwardly as indicated by arrows 34, drawing the tissue with them to cause the tissue about the arteriotomy to become pursed toward a closed configuration suggested in phantom at 36. While retaining the tissue in that configuration, the guidewire 16 then may be removed and a closure device (not shown) may be inserted through the lumen 14 of the central tube 12. Upon operation of the closure device to secure the arteriotomy in its closed position, all of the devices may be removed.

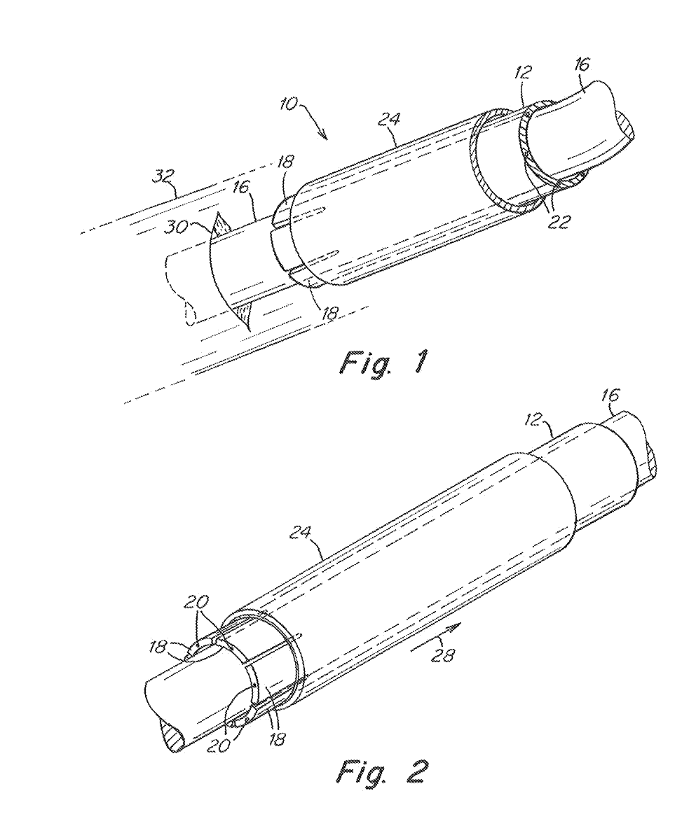

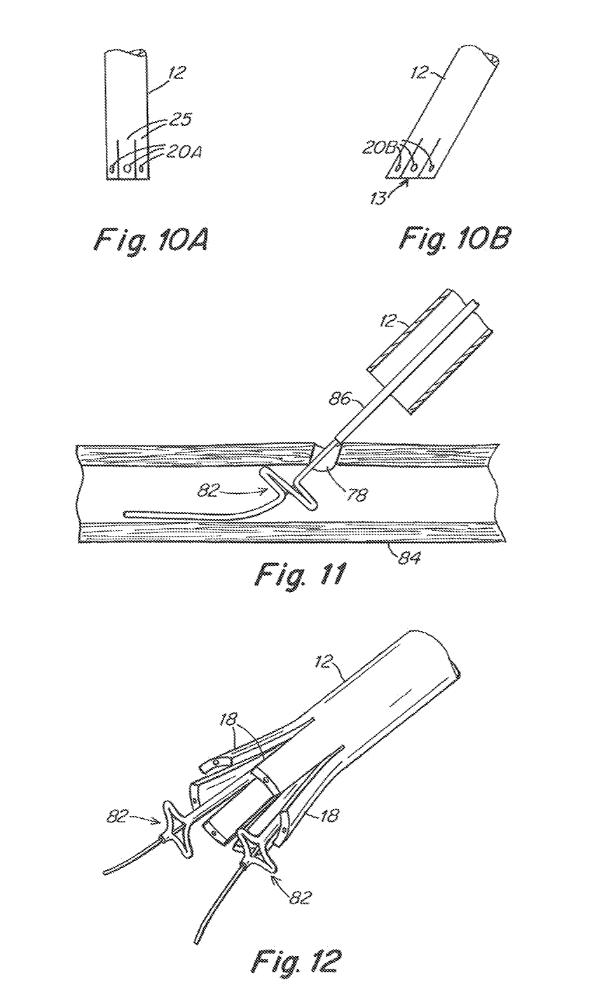

The suction ports 20 may be located in various configurations. FIGS. 2 and 3 show the ports as being exposed on the distal faces of the fingers. It may be desirable, in some cases, to locate the suction ports on the outer faces of the fingers as suggested at 20A in FIGS. 10A and 10B. FIG. 10B illustrates diagrammatically a configuration for the distal end of the central tube 12 in which the end is beveled at 13. The bevel 13 angle may be selected to correspond to the approach angle (approximately 45°) of the device to the vessel The ports 20B in FIG. 10B also may be aligned along the bevel angle. It should be understood that approximation of the arteriotomy in the vessel wall does not necessarily require direct engagement with the vessel wall but, instead, may be accomplished by effecting a grip on the connective tissue, such as the femoral sheath, disposed about and connected to the outer surface of the vessel.

FIG. 6 illustrates one mode of construction for the mechanism. The central tube 12 may be a suitably flexible, extruded biocompatible plastic having lumens 40 extending through the wall of the tube 12. The fingers 18 may be formed integrally with the tube 12. Smaller tubes 42, formed from a material having sufficient elasticity and resilience are contained within the lumens 40 to communicate the suction to the ports 20 and to assist in biasing the fingers in their radially outward, splayed configuration. The suction tubes 42 may, for example, be formed from nitinol (TiNi) hypotube that may extend within the extruded lumens 40 in the wall of the central tube. The nitinol hypotubes may be heat set to a preformed shape (bent radially outward) so that they will bias the fingers 18 to the outward position of FIG. 3. It should be understood that, other resilient spring elements may be incorporated into or associated with the fingers 18 to bias them toward a radially outward configuration. The fingers may be contracted from the splayed to the contracted configuration (FIG. 1) by advancing the closure tube 24 distally, sliding it along the central tube. As the closure tube 24 is advanced distally from the configuration shown in FIG. 3, it overcomes the bias of the nitinol hypotubes so that continued advancement of the closure tube draws the fingers together. The nitinol hypotubes should enhance the ability for suction to be maintained at the suction outlets throughout such range of movement.

FIG. 6A illustrates another embodiment in which the central tube 124 is formed entirely from a shape memory alloy such as nitinol with the fingers 18A being heat set to be biased in the radially expanded, splayed configuration when at body temperature. Lumens 40A may be formed longitudinally through the wall of the tube by wire electrical discharge machining (EDM). For example, using a 0.31 inch EDM electrode would enable fabrication of a finished hole of the order of 0.035 inch in the tube wall with a depth of up to about six inches. Should it be necessary to make the device longer, additional tubes could be fabricated and joined end-to-end with the vacuum lumens 40A aligned. The tube may, for example, be of the order of about 0.23 inch diameter with a wall thickness of about 0.072 inch and an inner diameter of about 0.09 inch. The distal end of the nitinol central tube 12A may be formed similarly to the above-described embodiments to have a plurality of fingers with vacuum ports at or about the distal ends or outer surfaces of the fingers. The nitinol should be treated so that the fingers 18A are biased to the radially expanded configuration yet are returnable to the non-expanded configuration by advancement of the closure tube 24.

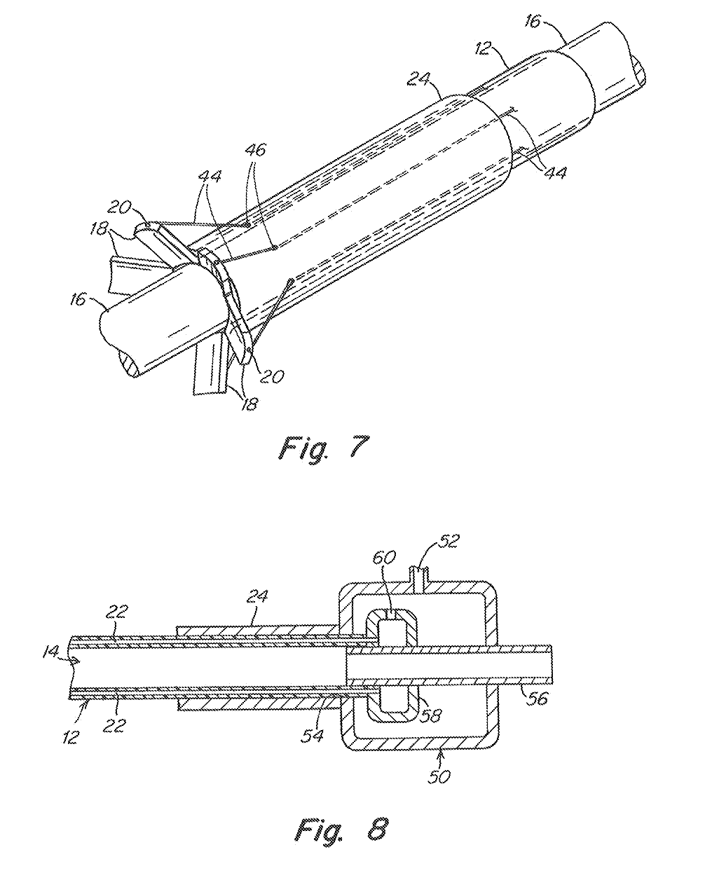

FIG. 7 illustrates another configuration for deploying and contracting the tissue retention fingers 18. In this embodiment, the fingers 18 are expanded radially by pull wires such as filaments 44 secured to the outer ends of the fingers 18. The filaments may be formed from polymers such as polypropylene, polyethylene terephthalate or nylon or from metals such as nitinol, nickel-cobalt-chromium-molybdenum superalloy, stainless steel or the like. Filaments 44 may be attached to the fingers 18 by knotting, laser welding or other appropriate means for attachment as will be familiar to those skilled in the art. The filaments may be of a fine diameter of the order of about 0.0015 to about 0.005 inch. The filaments may extend through apertures 46 in the closure tube 24 and may extend proximally to a control point at the proximal end of the system. Pulling on the filaments causes the fingers to pivot or flex about their roots 25. (See, e.g., FIG. 10A.) Alternately, the filaments may be secured directly to the closure tube 24 so that retraction of the closure tube 24 will tension the filaments 44 to expand the fingers and distal advancement will close the fingers. The fingers may be returned to a contracted configuration by advancing the closure tube 24 in a distal direction.

FIG. 8 illustrates one arrangement at the proximal end of the device by which the device may be controlled. The proximal end of the device may include a sealed housing 50 secured to the proximal end of the closure tube 24. The housing 50 includes a suction port 52 that is connectible to a source of suction (not shown). The central tube 12 extends through a slidably sealed opening 54 in the housing and includes a proximally extending extension tube 56 adapted to receive a guidewire or a delivery device for the arteriotomy closure. The device also includes a manifold 58 that is secured to the central tube 12, is contained in the housing 50 and communicates with the proximal ends of the suction lumens 22 in the central tube 12. The manifold 58 also is in communication with the interior of the housing 50 by one or more ports 60 so that the suction will be communicated from the housing to the distal suction ports 20 through the suction lumens 22. The housing 50 and closure tube 24 thus are movable together with respect to the central tube 12 and the manifold 58 to cause or enable the fingers 18, 18A at the distal end of the device to expand from a contracted to a radially deployed configuration and to be retracted.

FIGS. 9A-9D illustrate, diagrammatically, the manner in which the device may cause approximation of the edges of the arteriotomy without directly engaging the vessel wall. After the vascular procedure has been completed, a guidewire 16 is placed (or left in place) through the tissue track 70 and into the vessel and the introducer then is withdrawn. The tissue track 70 extends through the skin 72, subcutaneous and connective tissue 74, including fascia and the femoral sheath which are attached to the outer adventitia of the vessel 76. FIG. 9B illustrates the distal end of the device advanced along an indwelling guidewire 16 through the track 70 as it approaches the connective tissue 74 disposed about the region of the arteriotomy 78. When the device is positioned proximally of the arteriotomy, the closure tube 24 is retracted to enable or cause the fingers to be deployed radially outward into engagement with the connective tissue 74 about and proximally of the puncture in the vessel wall (FIG. 9C). Suction then is applied to cause the connective tissue 74 to be drawn securely against the suction ports. The connection between the connective tissue and the vessel wall is such that the connective tissue can be drawn while maintaining its attachment to the vessel wall. Thus, as the connective tissue 74 is gripped by the fingers 18 and when the fingers are drawn inwardly by actuation of the control sheath 24, they will draw the connective tissue 74 together and that, in turn, draws the edges 80 of the arteriotomy 78 toward each other by reason of the connection (FIG. 9D). With the connective tissue so held by the device, the guidewire may be withdrawn (FIG. 9D) and a closure device (not shown) adapted to make a more permanent closure may be advanced through the central tube 12 to engage with the puckered connective tissue. After removal of closure devices, a short duration of external pressure may be desirable as a precaution.

The invention also may be practiced in conjunction with a stabilizing device, for example, of the type described in U.S. Pat. No. 6,767,356 (Kanner). As shown in FIG. 11, wire-like stabilizers 82 may extend through the central tube 12. The distal ends of the stabilizers are configured to be placed through the arteriotomy 78 into the lumen of the vessel 84. The stabilizers 82, which are inserted in a linear configuration, then are operated to an enlarged configuration, as suggested in FIG. 11 or 12 so that they cannot be withdrawn through the arteriotomy 78. The stabilizers 82 thus provide a stable platform with which the central tube 12 can be held in centered position over the region of the arteriotomy. The stabilizers may include a guide 86 that may be secured to the sheath, and through which the stabilizer 82 may be advanced or retracted when in its linear, non-deployed configuration. Reference is made to the Kanner '356 patent for additional details concerning various constructions and embodiments of the stabilizing system, which are incorporated by reference herein, in their entirety.

It should be understood that the foregoing description of the invention is intended merely to be illustrative thereof and that other embodiments and modifications may be apparent to those skilled in the art without departing from the scope of the invention and equivalents thereof.

1. A device for closing an arteriotomy comprising:
an elongate central tube having proximal and distal ends, the distal end being configured to movable between radially contracted and radially expanded configurations;
a plurality of gripping elements adapted to contact and grip tissue by its surface, the gripping elements being disposed on the radially expandable distal end of the central tube, whereby the gripping elements can be located circumferentially about the tube;
means for controlling the distal end of the central tube to selectively expand or contract the radial positions of the gripping elements;
whereby when the gripping elements are in their radially expanded positions, they may be operated to effect a grip on the tissue;
means for returning the gripping elements to their contracted position while maintaining grip on the tissue whereby the tissue may be pursed together;
the central tube having a lumen to provide access to the pursed tissue while held by the contracted gripping elements,

2. The device as defined in claim 1 wherein the gripping elements comprise suction ports.

3. The device as defined in claim 1 wherein the radially expandable distal end of the tube comprises a plurality of fingers integral with the tube, the fingers being movable relative to the body of the tube to locate the gripping elements radially spaced from the tube.

4. The device as defined in claim 3 wherein the fingers are resiliency biased radially outwardly; and
a closure tube slidably disposed on the central tube, the closure tube being slidable along the central tube and engageable with radially extending fingers thereby to urge the fingers from their radially extended to their contracted positions.

5. The device as defined in claim 4 further comprising;
resilient spring elements mounted to the fingers, the spring elements being configured to bias the fingers in a radially outward deployed configuration.

6. The device as defined in claim 3 wherein the means for expanding the radial position of the gripping elements comprises a pull wire associated with each of the fingers, the pull wires being arranged to draw the fingers from their contracted to their radially extended positions.

7. The device as defined in claim 6 further comprising a closure tube slidably disposed on the central tube, wherein the pull wires are attached to the closure tube to draw the fingers to their radially expanded configuration in response to retraction of the closure tube.

8. The device as defined in claim 2 further comprising a plurality of lumens extending longitudinally through the wall of the central tube, each of the lumens being in communication with a suction port.

9. The device as defined in claim 3 further comprising:
a plurality of resilient tubes extending longitudinally through the wall of the central tube, each of the resilient tubes communicating with one of the suction ports and extending through a finger associated with the suction port, each resilient tube being arranged to bias its associated finger in its radially outward configuration.

10. The device as defined in claim 9 wherein the resilient tubes are formed from nitinol.

11. The device as defined in claim 2 wherein the suction ports are formed on the distal ends of the fingers.

12. The device as defined in claim 2 wherein the suction ports are formed on the sides of the fingers.

13. The device as defined in claim 3 wherein the distal end of the central tube and ends of the fingers are beveled at an angle to a longitudinal axis of the central tube and wherein the gripping elements lie along a plane generally paralleling the bevel.

14. A method for closing an arteriotomy comprising;
providing a plurality of tissue gripping elements on a distal end of a central tube, the distal end of the tube being configured to be movable to selectively position the gripping elements between radially contracted and radially expanded configurations;
disposing the gripping elements in surrounding relation, to the arteriotomy;
actuating the gripping elements to cause them to grip tissue surface without piercing the tissue;
thereafter drawing the gripping elements, and the tissue gripped thereby, together to purse the tissue together; and
while holding the tissue about the arteriotomy in pursed configuration, applying a securing means to the tissue to permanently secure the arteriotomy closed; and
thereafter releasing the gripping elements from the tissue.

15. The method as defined in claim 14 where the gripping elements comprise suction ports and the step of actuating the gripping elements comprises applying suction to the suction ports.

16. The method as defined in claim 14 further comprising providing stabilizers to facilitate positioning of the central tube with respect to the arteriotomy and, placing the stabilizers through the arteriotomy and expanding them to an increased profile to resist withdrawal while in their expanded state.
